(19) United States
(12) Plant Patent Application Publication
(10) Pub. No.: US 2003/0074704 P1
(43) Pub. Date: Apr. 17, 2003

(54) Clematis variety 'Evirida'

(76) Inventors: Raymond J. Evison, St. Sampsons (GB); Mogens Olesen, Fredensborg (DK)

Correspondence Address: Mogens N. Olesen, 620 S. Front Street, Central Point, OR, 97502, US

(21) Appl. No.: 09/863,123

(22) Filed: May 22, 2001

(30) Foreign Application Priority Data
Mar. 26, 1997 (EP) 97/0412

(51) Int. Cl. A01H 5/00

(52) U.S. Cl. PLT/228

(57) ABSTRACT

A new evergreen Clematis cultivar which has long continuous flowering season over which it produces attractive white flowers with a hint of green and a green center. This new and distinct variety has shown to be uniform and stable in the resulting generations from asexual propagation.

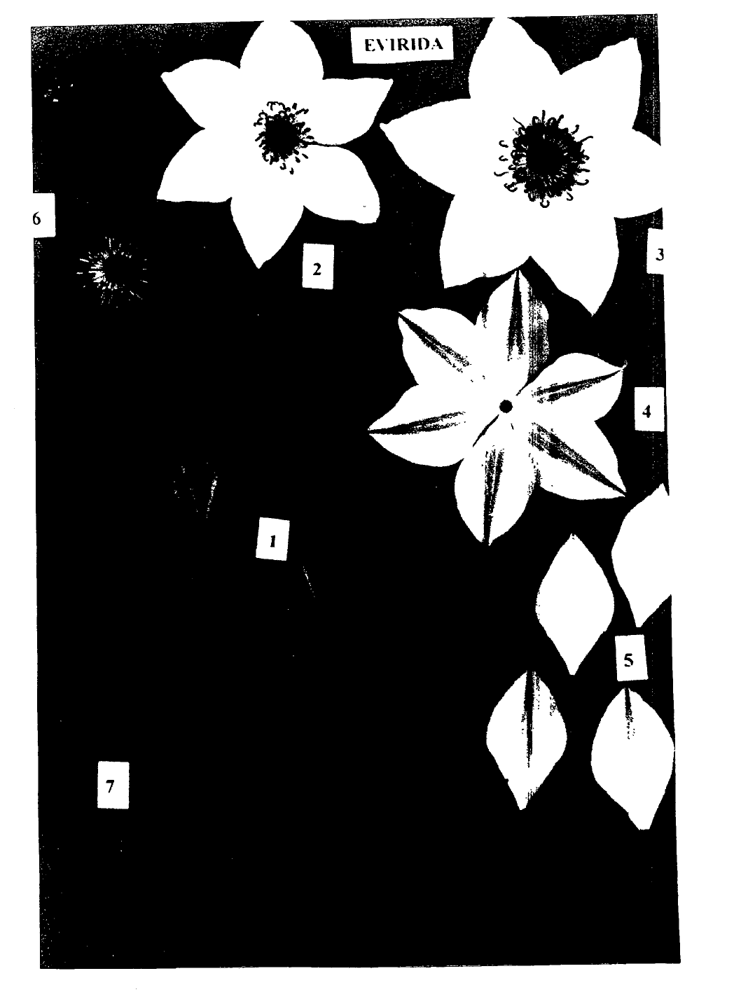

### SUMMARY OF THE DISCOVERY

The present discovery constitutes a new and distinct variety of Clematis which was discovered in a cultivated area. The mutation resulted from a naturally occurring whole plant mutation of unknown causation on a Clematis plant growing in a cultivated area. The resulting mutation was evaluated and asexually propagated in a controlled environment. The new variety is named ‘EVIrida’.

The clematis plant of the present discovery has a unique combination of characteristics which are outstanding in the new variety and which distinguish it from the original clematis, C. Florida The unique qualities that this variety has:

1. An attractive flat flower form with an open center.

2. Broad, slight overlapping tepals giving the flower a full form.

3. Flower has a central boss of green sterile styles.

4. Continuous flowering period, from May to October.

This combination of qualities was lacking in clematis varieties that were in commercial cultivation and the qualities have been substantially achieved in the new variety.

‘EVIrida’ was selected by Raymond J. Evison and Mogens N. Olesen in their Clematis development program in Domarie Vineries Les Sauvagees, St. Sampsons, Guernsey, Channel Islands, United Kingdom in 1993.

Asexual reproduction of ‘EvIrida’ by cuttings was first done by Raymond J. Evison in Domarie Vineries Les Sauvagees, St. Sampsons, Guernsey, Channel Islands, United Kingdom in 1993. This initial and subsequent propagations have demonstrated that the characteristics of ‘EVIrida’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers and leaves of the new variety, with different flowers shown in various stages of maturity. The flower form, flower colors, flower buds, floral parts, flower pedicels and the cultivar's foliage are depicted.

Specifically illustrated in SHEET 1:

1. Stem showing branching and the attachment of leaves, nodes, and a bud;

2. Flower buds and pedicels;

3. Partially open flower bud;

4. Flowers. The upper flower being completely open, the lower flower being less developed;

5. Reverse side of flower;

6. Single leave; and

7. Petiolule and leaflets.

### DETAILED DESCRIPTION OF THE VARIETY

The following is a detailed description of ‘EVIrida’, as observed in its growth throughout the flowering period in glasshouses at Domarie Vineries Les Sauvagees, St. Sampsons, Guernsey, Channel Islands, United Kingdom. Certain phenotypical characteristics of the variety may vary under different environmental, cultural, agronomic, seasonal, and climatic conditions. Color references are made using The Royal Horticultural Society (London, England) Colour Chart, 1995, except where common terms of color are used. For a comparison, the nearest existing Clematis variety is C. Florida. Chart 1 details the physical characteristics of the applicant and the comparison varieties.

CHART 1

| | ‘EVIrida’ | C. Florida |
|---|---|---|
| Filaments | Long, white | Short, |
| | | purple. |
| Center | Very open, | Compact, |
| Form. | filaments held | closed. |
| | horizontal. | |
| Center | Green (Exposed | Dark purple |
| Color | styles) | (Anthers, anclose |
| | | stigmas) |
| Tepal | Flat | Recurved |
| Recurvature | | |

Parent: Whole plant mutation.

Classification:

Botanical.—Clematis L.

Commercial.—Double flowered compact cultivar.

### FLOWER AND FLOWER BUD

Blooming habit: Continuous.

Flower bud:

Size.—20-30 mm in length.

Bud form.—Elliptical.

Bud color.—Yellow-Green Group 145B at ¼ opening.

Tepals.—Yellow-Green Group 149D.

Peduncle.—Surface: Smooth. Length: 90-120 mm average length. Color: Medium green. Green Group 143C. Strength: Horizontal.

Borne.—Singly.

Flower bloom:

Size.—Medium. 90 mm on average.

Form.—Flat. Completely open, outer tepals are flat.

Color.—Upon opening, the upper surface is Yellow-Green Group 149D. Upon opening, the reverse side is Yellow-Green Group 149D,with a central streak running the length of the tepal, Yellow-Green Group 144B. After opening, the upper surface is White Group 157D. After opening, the reverse surface is White Group 157D, with a central streak running the length of the tepal, Yellow-Green Group 149D. The general tonality of the open flower is White Group 157D.

Fragrance.—None.

Lasting quality on plant and as a cut flower.—Long.

Tepals:

Petalage.—Single. Average range: 6.

Shape.—Individual tepal shape is elliptical.

Cross section.—Flat.

Undulation of margin.—Weak.

Tepal apex.—Narrowly acute.

Recurvature of Tip.—None.

Persistence.—Outer tepals drop off cleanly.

Arrangement.—Tepals are arranged regularly.

Reproductive Organs:

Pollen.—None.

Anthers.—Size: Small, with long filaments. Color: Light reddish-brown in color. Arrangement: Regular

Filaments.—Color: White.

Stigmas.—None.

Styles.—Color: Green.

Ovaries.—Normal.

### PLANT

Plant form: Climbing and spreading.

Plant growth: Moderately vigorous.

Height: When trellised, one seasons growth attains 2.0-3.0 meters.

Hardiness: Trials to date show the variety hardy in USDA zones 6-9.

Stems:

Color.—Young wood: Green with intonations of brown. Older wood: Brown.

Internodes.—Cylindrical in cross section. Length 130-180 mm.

Petioles.—Size: Average length: Petiolus=30-60 mm Petiolule=0-10 mm Color: Medium green. Clasping: Clasps by leaf petiolus.

Bark.—Young wood: Smooth. Older wood: Smooth.

Plant foliage: Evergreen. Varies between: a) Trifoliate; b) 3 trifoliate leaflets arranged in trifoliate form; c) 5 leaflets arranged in pinnate form.

Leaf size.—Small. Length 20-40 mm.

Abundance.—Average.

Color.—Top: Green Group 137A. Bottom: Green Group 137C. New growth: Internodes green. Foliage green. Older growth: Internodes dark green, turning brown. Foliage dark green.

Plant leaves and leaflets:

Stipules.—Absent.

Petiole.—Length: 30-60 mm. Underneath: Without prickles.

Leaf edge.—Terminal leaflet margin is normally entire but sometimes undulated and/or with rounded lobes.

Shape.—Base of leaflet: Rounded. Apex of leaflet: Acute.

Texture.—Thin. Upper side: Glabrous. Lower side: Ribbed.

Surface.—Matte Finish.

Disease resistance: Subject to any disease which normally attacks the species.

Additional Information: After tepals drop off stamens fold back completely, revealing green boss of sterile styles, some of which by this stage have a miniature petaloid-stamen-like appearance. This is unique—no other clematis displays this.

1. A new and distinct variety of clematis plant, substantially as herein illustrated and described, as a distinct and novel clematis variety due to its abundant white flowers, excellent flower longevity, long recurrent flowering period from early summer through autumn, and compact growth making it suitable for nursery production in containers.
